(19) United States
(12) Patent Application Publication
(10) Pub. No.: US 2025/0182909 A1
(43) Pub. Date: Jun. 5, 2025

(54) INFORMATION PROCESSING METHOD

(71) Applicant: NEC Corporation, Minato-ku, Tokyo (JP)

(72) Inventor: Yuki Kosaka, Tokyo (JP)

(73) Assignee: NEC Corporation, Minato-ku, Tokyo (JP)

(21) Appl. No.: 18/840,557

(22) PCT Filed: Mar. 14, 2022
(86) PCT No.: PCT/JP2022/011297

§ 371 Date: Aug. 22, 2024

(51) Int. Cl. G16H 50/70; G06F 18/2321; G16H 10/60

(52) U.S. Cl. CPC G16H 50/70; G06F 18/2321; G16H 10/60

(57) ABSTRACT

An information processing apparatus of the present invention includes: a clustering unit that classifies combination information representing a combination of a plurality of types of measures performed on a target person for each time, as any one of a plurality of clusters set in advance; a first model generating unit that generates a first model based on condition information representing a condition of the target person and the cluster as which the combination of the plurality of types of measures performed on the target person is classified for each time, the first model outputting the cluster for the condition information; and a second model generating unit that generates a second model based on the condition information, the cluster, and the combination information for each time, the second model outputting the combination information for information based on the condition information and the cluster.

### TECHNICAL FIELD

The present invention relates to an information processing method, an information processing apparatus, and a program.

### BACKGROUND ART

For treatment of patients, there is an increasing demand for personalized medicine, in which a treatment option is determined according to the condition of each individual patient. Personalized medicine aims to provide a treatment method that is tailored to each patient using individual's genetic information, medical information, and so forth. Then, the treatment method may include a plurality of treatments in combination, and optimum treatment selection according to the condition is required. Here, Patent Literature 1 describes selection of a treatment using a computer.

### CITATION LIST

### Patent Literature

Patent literature 1: Japanese Translation of PCT International Application Publication No. JP-T 2016-514291

### SUMMARY OF INVENTION

### Technical Problem

However, in a case where there are many selectable treatments, the number of combinations of a plurality of treatments becomes enormous, which makes it difficult to select an appropriate treatment option for a patient. In addition, as the combinations of treatment options is more, the cases of each combination of treatment options is fewer, which makes it difficult to even refer to past cases. Then, such problems occur in not limited to treatment, but also training, exercising, dieting, and the like, so that it is difficult to select a combination of a plurality of menus according to the condition of a person. That is to say, it is difficult to select a combination of a plurality of measures that can be implemented according to the condition of a target person.

Accordingly, an object of the present invention is to provide an information processing method that can solve the above issue that it is difficult to select a combination of a plurality of measures that can be implemented according to the state of a target person.

### Solution to Problem

An information processing method as an aspect of the present invention includes:
classifying combination information representing a combination of a plurality of types of measures performed on a target person for each time, as any one of a plurality of clusters set in advance;
generating a first model based on condition information representing a condition of the target person and the cluster as which the combination of the plurality of types of measures performed on the target person is classified for each time, the first model outputting the cluster for the condition information; and
generating a second model based on the condition information, the cluster, and the combination information for each time, the second model outputting the combination information for information based on the condition information and the cluster.

Further, an information processing apparatus as an aspect of the present invention includes:
a clustering unit that classifies combination information representing a combination of a plurality of types of measures performed on a target person for each time, as any one of a plurality of clusters set in advance;
a first model generating unit that generates a first model based on condition information representing a condition of the target person and the cluster as which the combination of the plurality of types of measures performed on the target person is classified for each time, the first model outputting the cluster for the condition information; and
a second model generating unit that generates a second model based on the condition information, the cluster, and the combination information for each time, the second model outputting the combination information for information based on the condition information and the cluster.

Further, a computer program as an aspect of the present invention includes instructions for causing an information processing apparatus to perform processes to:
classify combination information representing a combination of a plurality of types of measures performed on a target person for each time, as any one of a plurality of clusters set in advance;
generate a first model based on condition information representing a condition of the target person and the cluster as which the combination of the plurality of types of measures performed on the target person is classified for each time, the first model outputting the cluster for the condition information; and
generate a second model based on the condition information, the cluster, and the combination information for each time, the second model outputting the combination information for information based on the condition information and the cluster.

### ADVANTAGEOUS EFFECTS OF INVENTION

Configured as described above, the present invention can facilitate selection of a combination of a plurality of measures that can be implemented according to the condition of a target person.

### BRIEF DESCRIPTION OF DRAWINGS

FIG. 1 is a block diagram showing the configuration of an information processing apparatus in a first example embodiment of the present invention.

FIG. 2 is a view showing the aspect of data processing by the information processing apparatus disclosed in FIG. 1.

FIG. 3 is a view showing the aspect of data processing by the information processing apparatus disclosed in FIG. 1.

FIG. 4 is a view showing the aspect of data processing by the information processing apparatus disclosed in FIG. 1.

FIG. 5 is a view showing the aspect of data processing by the information processing apparatus disclosed in FIG. 1.

FIG. 6 is a view showing the aspect of data processing by the information processing apparatus disclosed in FIG. 1.

FIG. 7 is a view showing the aspect of data processing by the information processing apparatus disclosed in FIG. 1.

FIG. 8 is a view showing the aspect of data processing by the information processing apparatus disclosed in FIG. 1.

FIG. 9 is a view showing the aspect of data processing by the information processing apparatus disclosed in FIG. 1.

FIG. 10 is a flowchart showing the operation of the information processing apparatus disclosed in FIG. 1.

FIG. 11 is a flowchart showing the operation of the information processing apparatus disclosed in FIG. 1.

FIG. 12 is a view showing the aspect of data processing by an information processing apparatus in a second example embodiment of the present invention.

FIG. 13 is a view showing the aspect of data processing by the information processing apparatus in the second example embodiment of the present invention.

FIG. 14 is a block diagram showing the hardware configuration of an information processing apparatus in a third example embodiment of the present invention.

FIG. 15 is a block diagram showing the configuration of the information processing apparatus in the third example embodiment of the present invention.

FIG. 16 is a flowchart showing the operation of the information processing apparatus in the third example embodiment of the present invention.

### DESCRIPTION OF EXAMPLE EMBODIMENTS

### First Example Embodiment

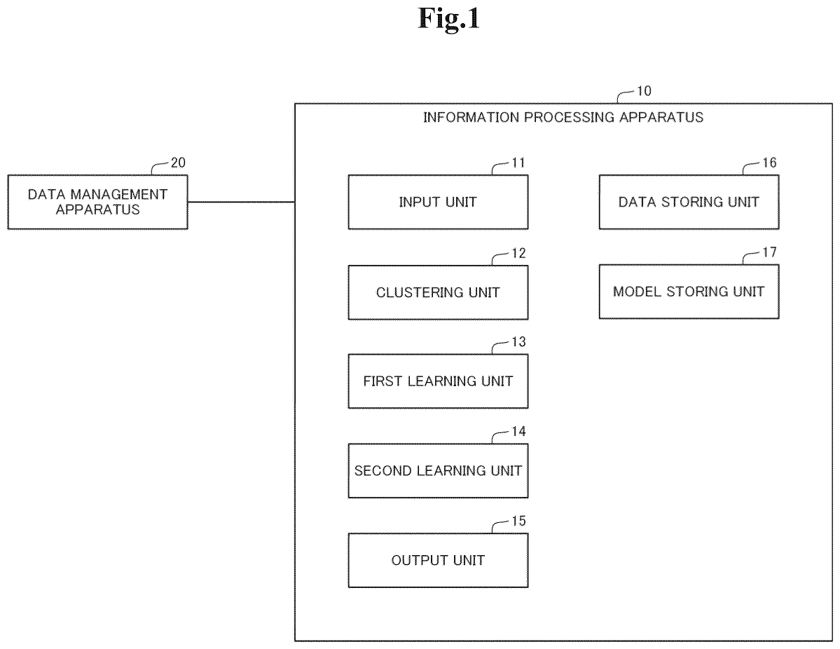

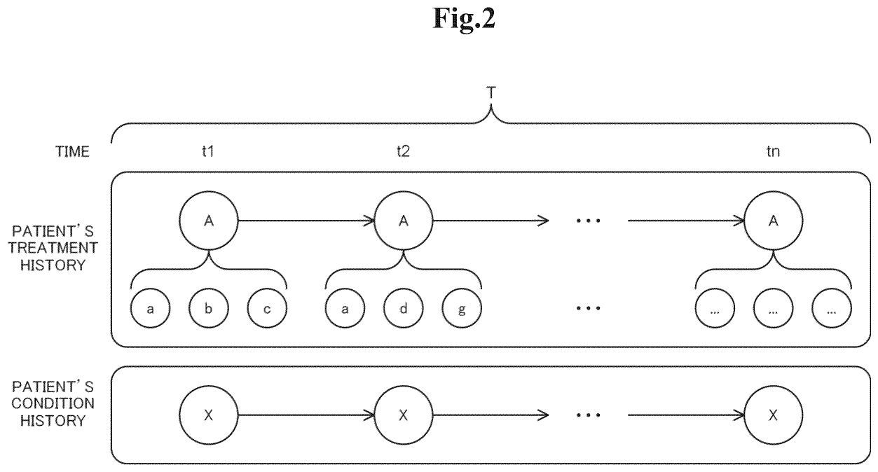

A first example embodiment of the present invention will be described with reference to FIGS. 1 to 11. FIG. 1 is a view for describing the configuration of an information processing apparatus, and FIGS. 2 to 11 are views for describing the processing operation of the information processing apparatus.

### [Configuration]

An information processing apparatus 10 of the present invention is used to, for treatment of patients, select a treatment method including a combination of a plurality of treatments according to the condition of each individual patient. Accordingly, the information processing apparatus 10 is also used to generate a model for selecting a treatment method including a combination of a plurality of treatments using existing patient data in advance. That is to say, the information processing apparatus 10 has a learning function to learn existing patient data and generate a model, and a selecting function to perform a process of selecting a future treatment method for a patient using the generated model. However, the information processing apparatus 10 of the present invention may also be used to select a combination of a plurality of measures (e.g., treatments, menus, actions, proposals) that can be implemented according to the condition of a target person in training, exercising, dieting and the like, not limited to treatment.

The information processing apparatus 10 is configured with one or a plurality of information processing apparatuses each including an arithmetic logic unit and a memory unit. Then, as shown in FIG. 1, the information processing apparatus 10 includes an input unit 11, a clustering unit 12, a first learning unit 13, a second learning unit 14, and an output unit 15, which are constructed by an arithmetic logic unit executing a program. The information processing apparatus 10 also includes a data storing unit 16 and a model storing unit 17 formed in the memory unit. The respective components will be described in detail below.

The input unit 11 requests patient data from a data management apparatus 20, accepts input of such patient data, and stores it into the data storing unit 16. Specifically, at the time of generating a model, the input unit 11 accepts and stores, as learning data, patient data that includes time (e.g., date) when a patient (target person) having already received a plurality of treatments received treatment, a treatment history (combination information) representing a combination of a plurality of types of treatments (measures) received by the patient, and a condition history (condition information) representing the condition of the patient at the time. Now, an example of the patient data will be described with reference to FIG. 2. In this view, it is assumed that a treatment history is A and a condition history is X at each of times t1, t2, . . . , tn during a period T. In this view, the treatment history A represents, for example, reception of a combination of three types of treatments, namely, treatments a, b, and c, at the time t1 and reception of a combination of three types of treatments, namely, treatments a, d, and g, at the time t2. Moreover, the condition history X represents, for example, the body temperature, blood pressure, heart rate, and so forth of the patient at each of the times. In addition, the condition history X may include information such as the age, height, weight, medical history, and so forth of the patient.

Further, at the time of performing a process of selecting a future treatment method for a patient, the input unit 11 requests and accepts input of patient data of the patient as selection data. For example, the input unit 11 accepts and stores patient data that includes current time (e.g., date) and a condition history (condition information) representing the condition of the patient at the time. As in the above description, the condition history includes information such as the body temperature, blood pressure, heart rate, age, height, weight, medical history, and so forth of the patient at each time.

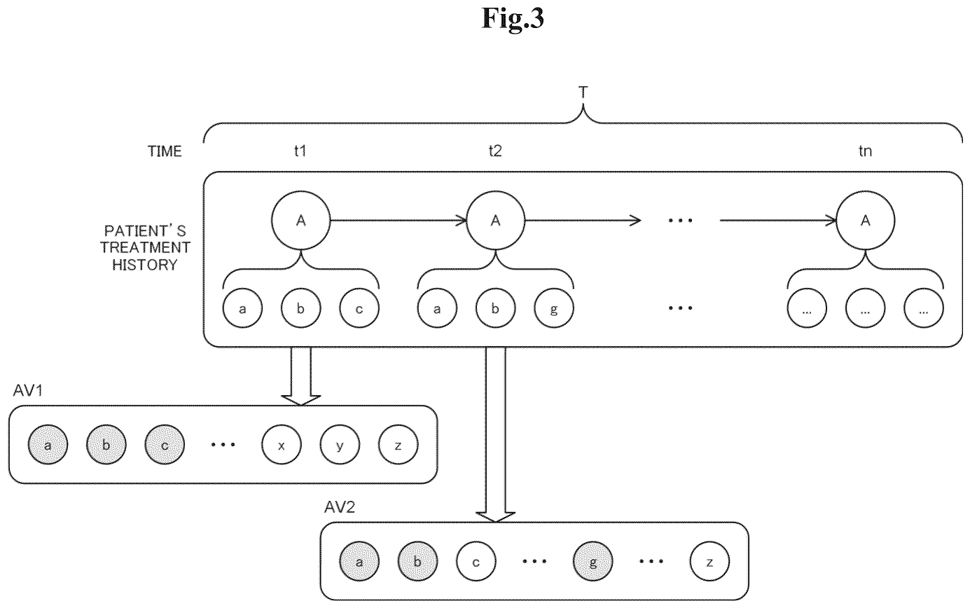

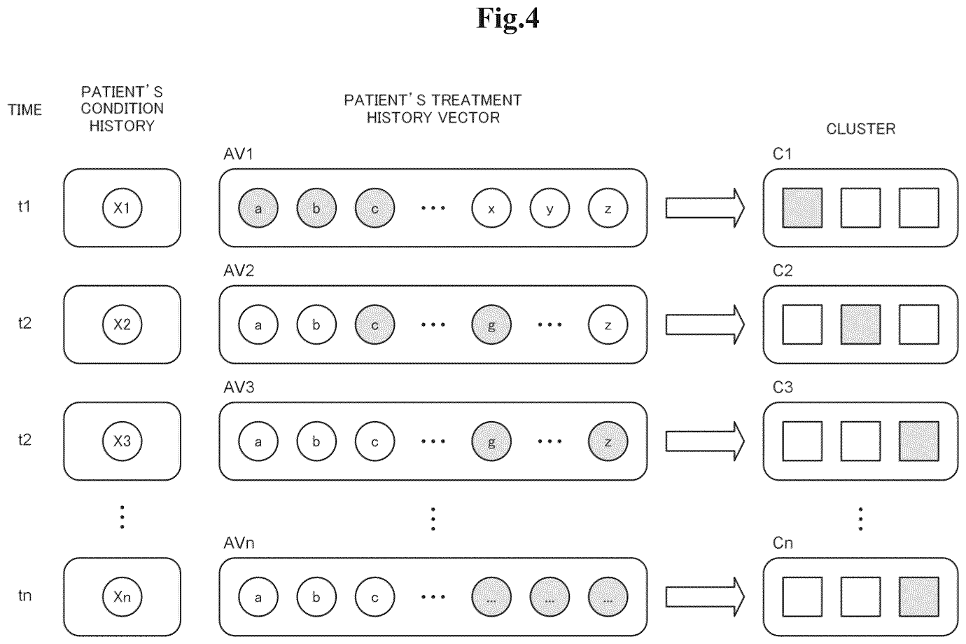

The clustering unit 12 performs a process of classifying a treatment history A included by patient data stored as learning data, as one of a plurality of clusters set in advance. Specifically, the clustering unit 12 converts a treatment history A to a vector representation having a treatment type as an element, and classifies it as one of a plurality of types of clusters set in advance according to the characteristics of the vector representation. Here, a specific example of processing by the clustering unit 12 will be described with reference to FIGS. 3 and 4.

First, a process of converting the treatment history A of a patient to a vector representation will be described with reference to FIG. 3. FIG. 3 shows the treatment history A of a certain patient during a period T. For example, in a case where the total number of selectable treatment types is 26, from a to z, the clustering unit 12 converts it to a treatment history vector AV, in which the total number of elements is 26, an element of a treatment having been performed has a value of 1 (gray circle in FIG. 3) and an element of a treatment having not been performed has a value of 0 (white circle in FIG. 3). As an example, in a case where the treatment history A at time t1 is a combination of three types of treatments, a, b, and c, the clustering unit 12 converts it to a treatment history vector AV1, in which all the selectable treatments (a to z) are elements, elements of treatments a, b, and c having been performed have a value of 1, and elements having not been performed have a value of 0. In addition, in a case where the treatment history A at time t2 is a combination of three types of treatments, a, b, and g, the clustering unit 12 converts it to a treatment history vector AV2, in which all the selectable treatments (a to z) are elements, elements of treatments a, b, and g having been performed have a value of 1, and elements having not been performed have a value of 0.

Next, referring to FIG. 4, a process of classifying patient's treatment history vectors AV into clusters C is performed. Here, for example, the clusters C each include three elements, one of which has a value of 1 (gray square in FIG. 4) and the other elements have a value of 0 (white square in FIG. 4). Thus, the number of types of clusters C is set to three, which is less than 26 that is the number of types of treatments mentioned above. However, the number of treatments and the number of clusters are not limited to the above numbers. The clustering unit 12 classifies a treatment history vector AV as one of the three clusters C according to the characteristics of the vector array of the treatment history vector AV, for example, according to the distribution of elements whose value is 1 in the treatment history vector AV. As an example, since the treatment history vector AV1 has elements having a value of 1 concentrated in the left region, the clustering unit 12 classifies it as cluster C1, in which a first element on the left side has a value of 1. However, the clustering unit 12 may classify treatment history vectors AV into clusters C by any method.

As described above, the clustering unit 12 converts treatment histories A at the respective times of a patient to treatment history vectors AV and classifies them into clusters C. Then, the clustering unit 12 converts treatment histories A at each time included by patient data of a plurality of patients acquired as learning data to treatment history vectors AV and classifies them into clusters C.

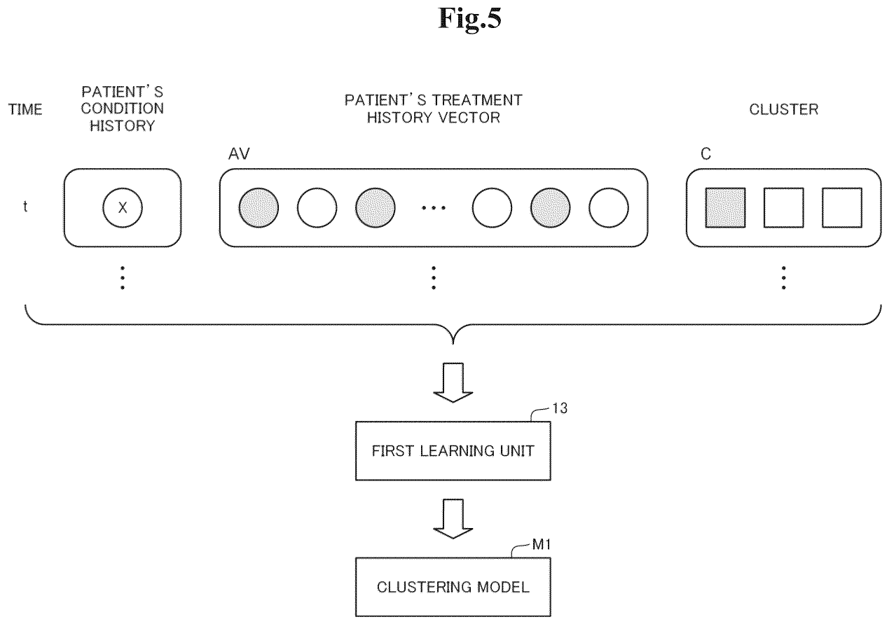

The first learning unit 13 (first model generating unit) performs machine learning using a condition history X of a patient and a cluster C as which a treatment history A of treatment performed on the patient is classified at each time, and generates a clustering model M1 (first model) that outputs a new cluster from a new condition history of the patient. Now, a process of generating a clustering model M1 will be described with reference to FIG. 5. The first learning unit 13 inputs a condition history X of a patient, a treatment history vector AV of the patient, and a cluster C at each time t into the first learning unit 13, learns the relation of them, and generates a clustering model M1 that outputs a new cluster in response to input of a new condition history. Then, the first learning unit 13 stores the generated clustering model M1 into the model storing unit 17.

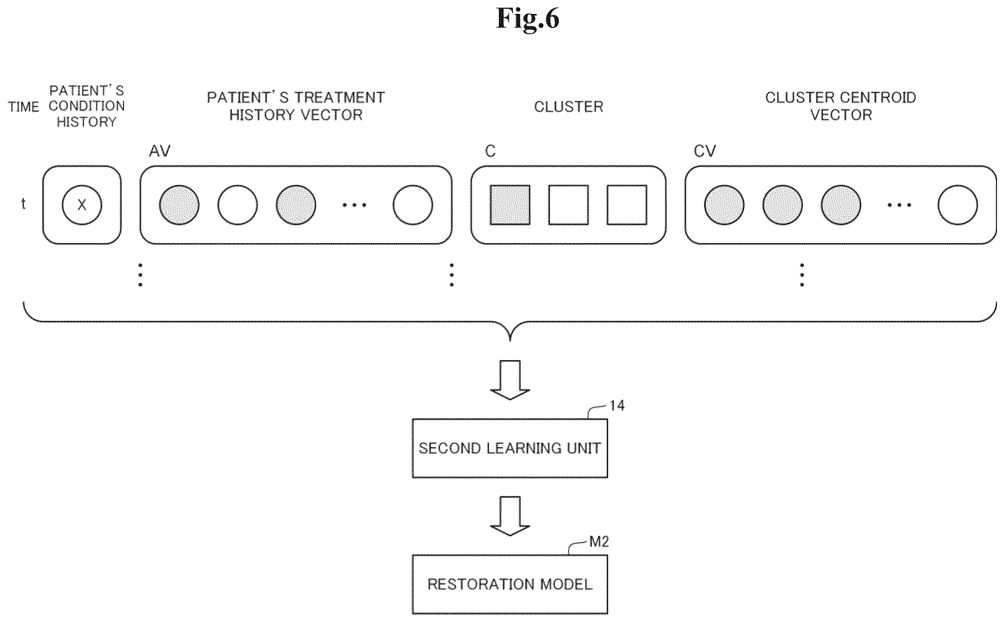

The second learning unit 14 (second model generating unit) performs machine learning using a condition history X of a patient, a treatment history vector AV of a treatment history A performed on the patient, and a cluster C as which the treatment history A is classified at each time, and generates a restoration model M2 (second model) that outputs a new treatment proposal (combination information). Now, a process of generating a restoration model M2 will be described with reference to FIG. 6. Specifically, the second learning unit 14 inputs a condition history X of a patient, a treatment history vector AV of the patient, a cluster C, and a cluster centroid vector CV (cluster characteristics information) representing the characteristics of the cluster C as a vector whose elements are a plurality of types of treatments at each time t, and thereby learns the relation of them. Then, as a result of the learning, the second learning unit 14 generates a restoration model M2 that outputs a new treatment proposal in response to input of a new condition history of the patient and a cluster centroid vector corresponding to the cluster output by inputting the new condition history into the clustering model M1.

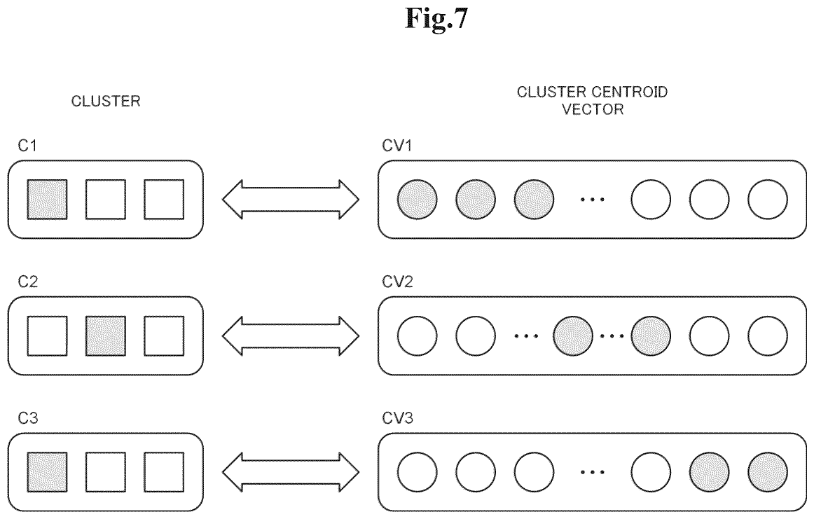

Here, the abovementioned cluster centroid vector is a vector in which all the treatments are elements and the value of an element corresponding to a treatment representing the characteristics of a cluster C is set to 1. For example, as shown in FIG. 7, a cluster centroid vector CV corresponding to each cluster C is set in advance. As an example, a cluster centroid vector includes 26 elements, which is the number of treatment types, and a cluster centroid vector CV1, in which elements having a value of 1 are concentrated in the left-side region, is associated with a cluster C1, in which the first element on the left side has a value of 1. In addition, as for the association of a cluster C and a cluster centroid vector CV as shown in FIG. 7, the cluster centroid vector CV can be associated with the identification information of the cluster C. Therefore, when performing machine learning as shown in FIG. 6, the abovementioned second learning unit 14 can input, not the vector information of a cluster C, but the identification information of the cluster C and input a cluster centroid vector corresponding thereto. That is to say, with input of a condition history X of a patient, a treatment history vector AV of the patient, the identification information of a cluster C, and a cluster centroid vector CV corresponding to the identification information of the cluster C at each time t, the second learning unit 14 generates a restoration model M2 that outputs a new treatment proposal.

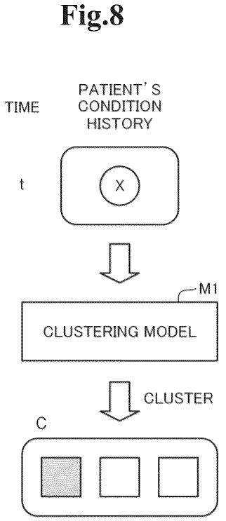

The output unit 15 is implemented when a process for selecting a future treatment method for a patient is performed. Specifically, the output unit 15 first acquires a condition history representing the current condition of a patient, which is included by the patient data of the patient accepted as selection data. As described above, a condition history includes information such as patient's body temperature, blood pressure and heart rate, and patient's age, height, weight and medical history. Then, as shown in FIG. 8, the output unit 15 reads out a clustering model M1 generated and stored in the model storing unit 17 as described above, and inputs the condition history of the patient into the clustering model M1. Then, a new cluster C corresponding to the input condition history is output from the clustering model M1, and the output unit 15 acquires the new cluster C.

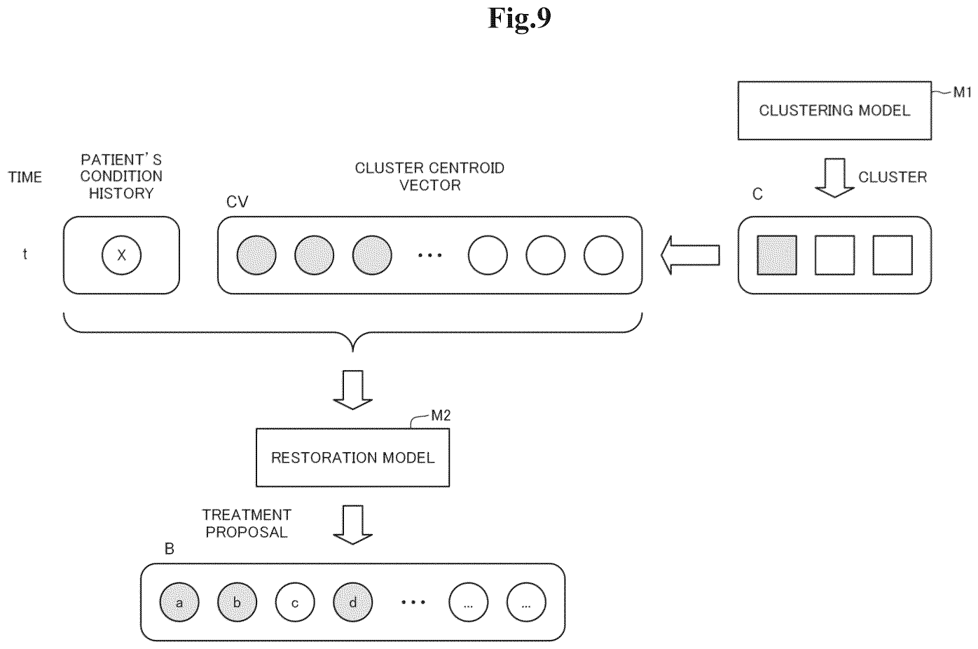

Next, the output unit 15 reads out a restoration model M2 generated and stored in the model storing unit 17 as described above, and inputs the condition history of the patient and information based on the new cluster C output from the clustering model M1 into the restoration model M2. Specifically, as shown in FIG. 9, the output unit 15 inputs a cluster centroid vector associated with the identification information of the new cluster C and the condition history of the patient into the restoration model M2. Then, from the restoration model M2, a treatment proposal B showing, in vector representation, a new combination of a plurality of treatments corresponding to the input condition history and cluster centroid vector, and the output unit 15 outputs this.

The output unit 15 extracts a plurality of treatments to be performed from the treatment proposal B in vector representation acquired as described above, and outputs them onto a display screen or the like. Thus, a person to whom the treatment proposal is shown can set a treatment method with reference to the treatment proposal.

### [Operation]

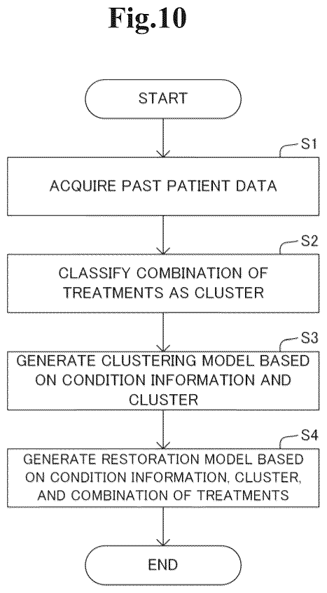

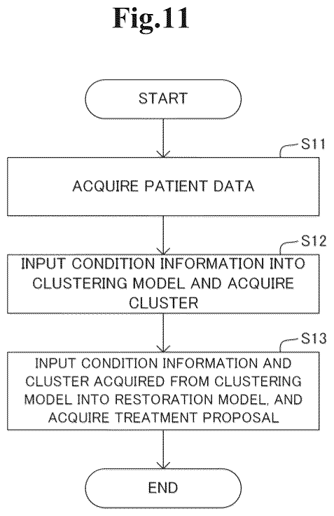

Next, the operation of the above information processing apparatus 10 will be described with reference to flowcharts of FIGS. 10 and 11. First, the operation of the information processing apparatus 10 when generating a model will be described with reference to FIG. 10. The information processing apparatus 10 requests past patient data from the data management apparatus 20 and acquires the patient data as learning data (step S1). As shown in FIG. 2, patient data as learning data includes time when a patient received a treatment, a treatment history representing the combination of a plurality of types of treatments that the patient received, and a condition history representing the patient's condition at the time. At this time, the information processing apparatus 10 acquires patient data at a plurality of times for each patient and patient data of a plurality of patients as learning data.

Next, the information processing apparatus 10 performs a process of classifying a treatment history A included by the patient data as one of a plurality of clusters set in advance (step S2). For example, first, as shown in FIG. 3, the information processing apparatus 10 converts a treatment history A at each time t to a treatment history vector AV expressed in vector representation having treatment types as elements. Then, as shown in FIG. 4, the information processing apparatus 10 classifies the treatment history vector AV as a corresponding cluster C according to the characteristics of the vector array thereof.

Next, as shown in FIG. 5, the information processing apparatus 10 performs machine learning using a condition history X and a cluster C of each patient and at each time, and generates a clustering model M1 (step S3). Thus, the clustering model M1 is trained to output a new cluster in response to input of a new condition history of a patient. Then, the information processing apparatus 10 stores the generated clustering model M1 into the model storing unit 17.

Next, as shown in FIG. 6, the information processing apparatus 10 performs machine learning using a condition history X, a treatment history vector AV of a treatment history A, a cluster C as which the treatment history A is classified, and a cluster centroid vector CV showing the characteristics of the cluster C as a vector whose elements are a plurality of types of treatments, of each patient and at each time, and generates a restoration model M2 (step S4). At this time, the cluster C used in the machine learning needs only identification information for identifying the cluster, and the cluster centroid vector CV associated in advance with the identification information of the cluster is used in the machine learning. Consequently, the restoration model M2 is trained to output a new treatment proposal in response to input of a new condition history of a patient and a cluster centroid vector corresponding to the cluster output by inputting the new condition history into the clustering model M1. Then, the information processing apparatus 10 stores the generated restoration model M2 into the model storing unit 17.

Next, the operation of the information processing apparatus 10 when selecting a treatment method will be described with reference to FIG. 11. The information processing apparatus 10 acquires current patient data from a patient as selection data (step S11). Patient data as selection data is a condition history representing the condition of a patient at current time t as shown in FIG. 8.

Next, the information processing apparatus 10 reads out the clustering model M1, and inputs the condition history of the patient into the clustering model M1. Then, as shown in FIG. 8, a new cluster C corresponding to the input condition history is output from the clustering model M1, and the information processing apparatus 10 acquires the new cluster C (step S12).

Next, the information processing apparatus 10 reads out the restoration model M2, and inputs the condition history of the patient and information based on the new cluster C output from the clustering model M1 into the restoration model M2. Specifically, as shown in FIG. 9, the information processing apparatus 10 inputs a cluster centroid vector associated with the identification information of the new cluster C and the condition history of the patient into the restoration model. Then, a treatment proposal B showing, in vector representation, a new combination of a plurality of treatments corresponding to the input condition history and cluster centroid vector is output from the restoration model M2, and the information processing apparatus 10 acquires the new treatment proposal B (step S13).

After that, the information processing apparatus 10 extracts a plurality of treatments to be performed from the acquired new treatment proposal B in vector representation, and outputs them onto a display screen or the like. Thus, a person to whom the treatment proposal is shown can set a treatment method with reference to the treatment proposal. As a result, in treatment for patients, it is possible to easily give a treatment proposal including a combination of a plurality of treatments according to the condition of each individual patient.

### Second Example Embodiment

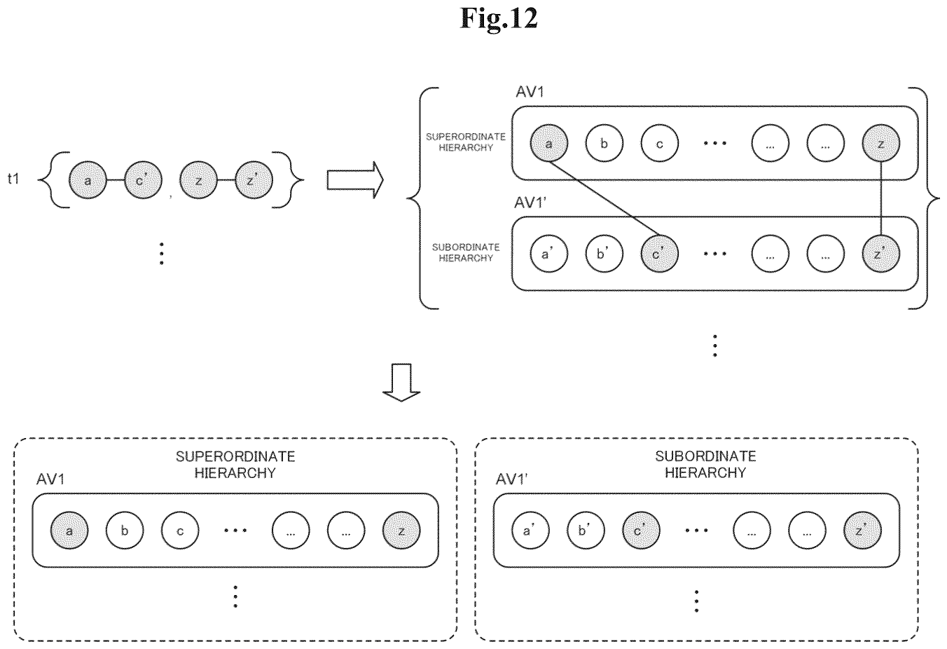

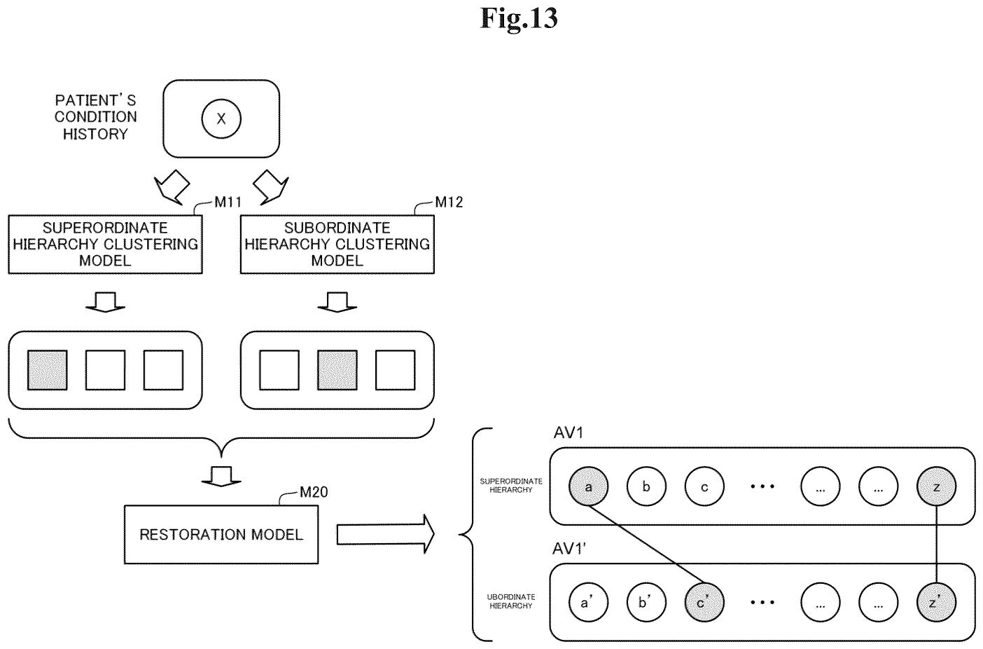

Next, a second example embodiment of the present invention will be described with reference to FIGS. 12 and 13. FIGS. 12 and 13 are views for describing the processing operation of the information processing apparatus 10 in the second example embodiment.

An information processing apparatus 10 in this example embodiment has the same configuration as the configuration shown in FIG. 1 described in the first example embodiment above. However, in this example embodiment, the data structures of a treatment history to be learned and a treatment proposal to be output from the model are different from those in the first example embodiment. Moreover, in this example embodiment, “treatment” will be reworded as “menu” and “patient” as “subject”, and an example will be given using a case of learning and proposing a combination of a plurality of menus performed in the fields of rehabilitation and training. However, “menu” described in this example embodiment may be the “treatment” described in the first example embodiment, or any “measure” (e.g., action, proposal) that can be implemented according to the condition of a subject. The configuration of the information processing apparatus 10 will be described below, focusing on the differences from the first example embodiment.

At the time of generating a model, the input unit 11 accepts and stores, as learning data, input of subject data including time (e.g., date) when a subject implemented a menu, a menu history (combination information) representing a combination of a plurality of types of menus (measures) implemented by the subject, and a condition history (condition information) representing the condition of the subject at the time. In this example embodiment, as shown in FIG. 12, a superordinate menu (first measure) set in a superordinate hierarchy (first hierarchy) and a subordinate menu (second measure) set in a subordinate hierarchy (second hierarchy) are set, and the superordinate menu in the superordinate hierarchy and the subordinate menu in the subordinate hierarchy are paired to form one menu, and a plurality of combinations of such paired menus become a menu history to be implemented at a predetermined time. For example, the superordinate menu set in the superordinate hierarchy may represent an exercise content such as “swimming” or “jogging”, and the subordinate menu set in the subordinate hierarchy may represent a time content such as an exercise duration like “1 hour” or exercise timing like “morning”. These are paired to form one menu (as an example, “swimming” for “1 hour”), and a plurality of paired menus are combined to form a menu history. In the example of FIG. 12, at time t1, a menu history is composed of a combination of two pairs of menus, the pair of superordinate menu a and subordinate menu c′ and the pair of superordinate menu z and subordinate menu z′.

The clustering unit 12 performs a process of classifying a menu history that is learning data as described above as one of a plurality of clusters set in advance. At this time, the clustering unit 12 first separates the menu history to the superordinate menu and the subordinate menu, and converts them to vector representations, respectively. For example, as shown in FIG. 12, the menu history is separated to a superordinate menu vector AV1 (first combination information) including superordinate menus of the superordinate hierarchy, and a subordinate menu vector AV1′ (second combination information) including subordinate menus of the subordinate hierarchy. At this time, the paired superordinate menu and subordinate menu are associated with each other between the superordinate menu vector AV1 and the subordinate menu vector AV1′. Then, the clustering unit 12 further classifies the superordinate menu vector AV1 separated to the superordinate hierarchy as one of superordinate hierarchy clusters C (first clusters) set in advance according to the characteristics of the vector array. Likewise, the clustering unit 12 classifies the subordinate menu vector AV1′ separated to the subordinate hierarchy as one of subordinate hierarchy clusters C (second clusters) set in advance according to the characteristics of the vector array. This process is performed for each superordinate hierarchy and each subordinate hierarchy by the method described with reference to FIG. 4 in the first example embodiment.

The first learning unit 13 (first model generating unit) generates clustering models M11 and M12 (first models) for each superordinate hierarchy and each subordinate hierarchy, as in the first example embodiment. Specifically, with learning data classified as the superordinate hierarchy as the target, the first learning unit 13 accepts a condition history X of a subject, a superordinate menu vector AV1 of the subject, and a superordinate hierarchy cluster C at each time t, learns the relation of them, and generates a superordinate hierarchy clustering model M11 that outputs a new superordinate hierarchy cluster in response to input of a new condition history. Likewise, with learning data classified as the subordinate hierarchy as the target, the first learning unit 13 accepts a condition history X of a subject, a subordinate menu vector AV1′ of the subject, and a subordinate hierarchy cluster C at each time t, learns the relation of them, and generates a subordinate hierarchy clustering model M12 that outputs a new subordinate hierarchy cluster in response to input of a new condition history. This process is performed for each superordinate hierarchy and for each subordinate hierarchy by the method described with reference to FIG. 5 in the first example embodiment. In this way, the first learning unit 13 generates the clustering model M11 corresponding to the superordinate hierarchy and the clustering model M12 corresponding to the subordinate hierarchy.

The second learning unit 14 (second model generating unit) performs machine learning using a condition history of a subject, a superordinate menu in a menu history, an superordinate hierarchy cluster as which the superordinate menu is classified, a subordinate menu in the menu history and a subordinate hierarchy cluster as which the subordinate menu is classified at each time, and generates a restoration model M20 (second model) that outputs a new menu proposal. Specifically, in this example embodiment, the second learning unit 14 performs machine learning, in addition to using the above information, using a superordinate cluster centroid vector that is associated with the identification information of a superordinate hierarchy cluster and represents the characteristics of the superordinate hierarchy cluster as a vector including superordinate menus as elements, and also using a subordinate cluster centroid vector that is associated with the identification information of a subordinate hierarchy cluster and represents the characteristics of the subordinate hierarchy cluster as a vector including subordinate menus as elements. Furthermore, the second learning unit 14 performs machine learning using, in addition to the above information, a superordinate menu vector AV1 and a subordinate menu vector AV1′ that represent a menu history in a state where paired superordinate menu and subordinate menu are associated with each other. Thus, the second learning unit 14 uses, as input, the condition history, the identification information and superordinate cluster centroid vector of the superordinate hierarchy cluster, the identification information and subordinate cluster centroid vector of the subordinate hierarchy cluster, and the superordinate menu vector AV1 and subordinate menu vector AV1′ in a state where paired superordinate menu and subordinate menu are associated with each other, and performs machine learning of the relation of them. Consequently, the second learning unit 14 generates a restoration model M20 that outputs a new menu proposal in response to input of a new condition history of a subject, a superordinate cluster centroid vector corresponding to a superordinate hierarchy cluster output as a result of inputting the new condition history into the superordinate clustering model M11, and a subordinate cluster centroid vector corresponding to a subordinate hierarchy cluster output as a result of inputting the new condition history into the subordinate clustering model M12.

The machine learning described above by the second learning unit 14 is performed by the same method as the method described with reference to FIG. 6 in the first example embodiment. However, the method in this example embodiment differs from the method shown in FIG. 6 in that clusters and cluster centroid vectors of the superordinate and subordinate hierarchies are input, and that a menu history includes a superordinate menu vector and a subordinate menu vector and is input in a state where paired superordinate and subordinate menus are associated with each other.

Further, at the time of performing a process of selecting a future menu for a subject, the input unit 11 accepts input of a condition history (condition information) representing the condition of the subject at that time as selection data.

Then, the output unit 15 reads out the superordinate hierarchy clustering model M11, the subordinate hierarchy clustering model M12, and the restoration model M20 that are generated as described above, and performs a process of selecting a future menu to be proposed to the subject from the condition history of the subject accepted as the selection data. Specifically, as shown in FIG. 13, the output unit 15 first inputs the condition history into the superordinate hierarchy clustering model M11 and the subordinate hierarchy clustering model M12. Then, the superordinate hierarchy clustering model M11 outputs a new superordinate hierarchy cluster corresponding to the input condition history, and the subordinate hierarchy clustering model M12 outputs a new subordinate hierarchy cluster corresponding to the input condition history, and the output unit 15 acquires the new superordinate hierarchy cluster and subordinate hierarchy cluster.

Subsequently, as shown in FIG. 13, the output unit 15 inputs the condition history, information based on the new superordinate cluster, and information based on the new subordinate cluster into the restoration model M20. At this time, specifically, similar to the processing described with reference to FIG. 9 in the first example embodiment, the output unit 15 inputs into the restoration model M20 a superordinate cluster centroid vector associated with the identification information of the new superordinate cluster, a subordinate cluster centroid vector associated with the identification information of the new subordinate cluster, and the condition history. Then, as shown in FIG. 13, the restoration model M20 outputs a menu proposal B representing, in vector representation, a new combination of a plurality of paired superordinate menus and subordinate menus corresponding to the input condition history, superordinate cluster centroid vector and subordinate cluster centroid vector, and the output unit 15 acquires this.

Thus, even if a combination of hierarchized menus (measures) is used as in this example embodiment, it is possible to easily make a proposal that combines a plurality of menus (measures) in accordance with the condition of each individual subject.

### Third Example Embodiment

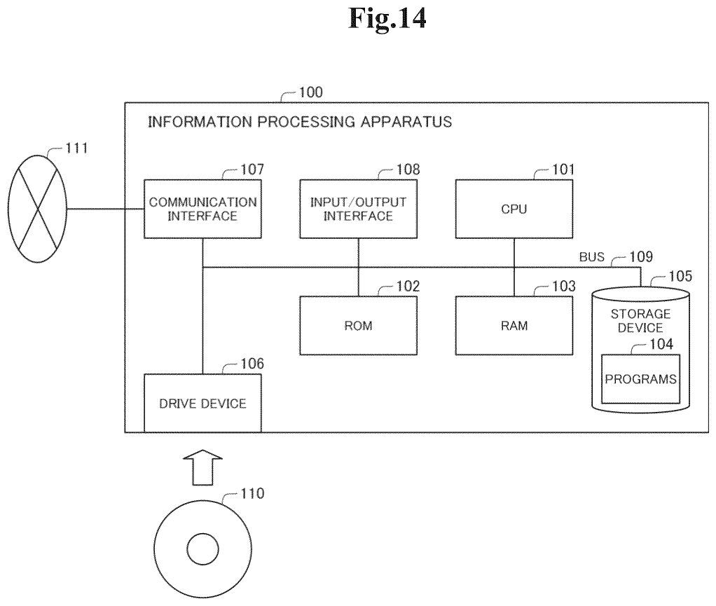

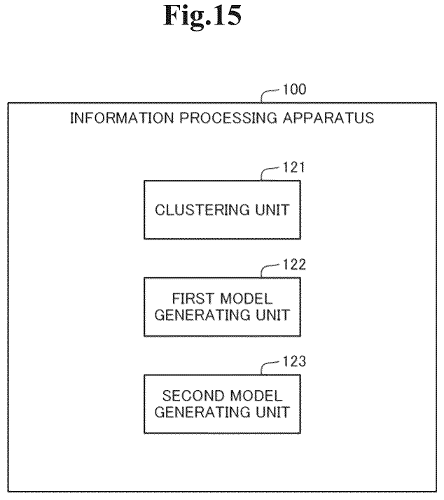

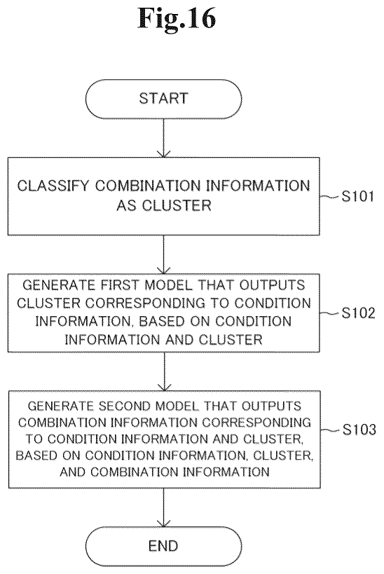

Next, a third example embodiment of the present invention will be described with reference to FIGS. 14 to 16. FIGS. 14 and 15 are block diagrams showing the configuration of an information processing apparatus in the third example embodiment, and FIG. 16 is a flowchart showing the operation of the information processing apparatus. In this example embodiment, the overview of the configurations of the information processing apparatus and the information processing method described in the above example embodiments is shown.

First, with reference to FIG. 14, the hardware configuration of an information processing apparatus 100 in this example embodiment will be described. The information processing apparatus 100 is configured with a general information processing apparatus, and as an example, has the following hardware configuration including;
a CPU (Central Processing Unit) 101 (arithmetic logic unit),
a ROM (Read Only Memory) 102 (memory unit),
a RAM (Random Access Memory) 103 (memory unit),
programs 104 loaded to the RAM 103,
a storage device 105 storing the programs 104,
a drive device 106 reading from and writing into a storage medium 110 outside the information processing apparatus,
a communication interface 107 connected to a communication network 111 outside the information processing apparatus,
an input/output interface 108 inputting and outputting data, and
a bus 109 connecting the respective components.

Then, by acquisition and execution of the programs 104 by the CPU 101, the information processing apparatus 100 can construct and include a clustering unit 121, a first model generating unit 122, and a second model generating unit 123 shown in FIG. 15. The programs 104 are, for example, stored in advance in the storage device 105 or the ROM 102, and loaded to the RAM 103 and executed by the CPU 101 as necessary. Moreover, the programs 104 may be provided to the CPU 101 via the communication network 111, or the programs 104 may be stored in advance in the storage medium 110 and read out by the drive device 106 and provided to the CPU 101. However, the abovementioned clustering unit 121, first model generating unit 122, and second model generating unit 123 may be constructed using dedicated electronic circuits for realizing such means.

FIG. 14 shows an example of the hardware configuration of the information processing apparatus serving as the information processing apparatus 100, and the hardware configuration of the information processing apparatus is not limited to the abovementioned case. For example, the information processing apparatus may be configured with part of the abovementioned configuration, such as without the drive device 106.

Then, the information processing apparatus 100 executes an information processing method shown in the flowchart of FIG. 16 using the functions of the clustering unit 121, the first model generating unit 122 and the second model generating unit 123 constructed by the program as described above.

As shown in FIG. 16, the information processing apparatus 100 executes processes to:
classify combination information representing a combination of a plurality of types of measures performed on a target person for each time, as one of a plurality of clusters set in advance (step S101);
based on condition information representing the condition of the target person and the cluster as which the plurality of types of measures performed on the target person is classified for each time, generate a first model that outputs the cluster for the condition information (step S102); and
based on the condition information, the cluster, and the combination information for each time, generate a second model that outputs the combination information for information based on the condition information and the cluster (step S103).

Configured as described above, the present invention enables output of new combination information of a plurality of measures based on a new condition of a target person by utilizing the generated first model and second model. For example, in treatment for patients, it is possible to easily make a treatment proposal that combines a plurality of treatments according to the condition of each individual patient.

It should be noted that the abovementioned programs can be stored and provided to a computer using various types of non-transitory computer-readable mediums. Non-transitory computer-readable mediums include various types of tangible storage mediums. Examples of non-transitory computer-readable mediums include a magnetic recording medium (e.g., floppy disk, magnetic tape, hard disk drive), a magneto-optical recording medium (e.g., magneto-optical disk), a CD-ROM (Read Only Memory), a CD-R, a CD-R/W, and a semiconductor memory (e.g., mask ROM, PROM (Programmable ROM), EPROM (Erasable PROM), flash ROM, and RAM (Random Access Memory)). The program may also be provided to a computer by various types of transitory computer-readable mediums. Examples of transitory computer-readable mediums include electrical signals, optical signals, and electromagnetic waves. The transitory computer-readable medium can provide the program to a computer via a wired communication path such as an electric wire or an optical fiber, or via a wireless communication path.

Although the present invention has been described above with reference to the example embodiments and so forth, the present invention is not limited to the above example embodiments. The configurations and details of the present invention can be changed in various manners that can be understood by one skilled in the art within the scope of the present invention. Moreover, at least one or more of the functions of the clustering unit 121, the first model generating unit 122 and the second model generating unit 123 described above may be executed by an information processing apparatus installed and connected in any location on the network, that is, may be executed by so-called cloud computing.

### Supplementary Notes

The whole or part of the example embodiments disclosed above can be described as the following supplementary notes. Below, the overview of the configurations of an information processing method, an information processing apparatus, and a program of the present invention will be described. However, the present invention is not limited to the following configurations.

### (Supplementary Note 1)

An information processing method comprising:
classifying combination information representing a combination of a plurality of types of measures performed on a target person for each time, as any one of a plurality of clusters set in advance;
generating a first model based on condition information representing a condition of the target person and the cluster as which the combination of the plurality of types of measures performed on the target person is classified for each time, the first model outputting the cluster for the condition information; and
generating a second model based on the condition information, the cluster, and the combination information for each time, the second model outputting the combination information for information based on the condition information and the cluster.

### (Supplementary Note 2)

The information processing method according to Supplementary Note 1, comprising
classifying the combination information as any one of the clusters a number of which is set to a smaller number than a number of the types of measures.

### (Supplementary Note 3)

The information processing method according to Supplementary Note 1 or 2, comprising
classifying the combination information in vector representation that contains the types of measures as elements, as any one of the clusters in accordance with a characteristic on the vector representation.

### (Supplementary Note 4)

The information processing method according to any one of Supplementary Notes 1 to 3,
generating the second model based on cluster characteristic information representing a characteristic of the cluster in addition to the condition information, the cluster, and the combination information for each time, the second model outputting the combination information for the condition information and the cluster characteristic information.

### (Supplementary Note 5)

The information processing method according to Supplementary Note 3, comprising
generating the second model based on cluster characteristic information in vector representation that represents a characteristic of the cluster and that contains the types of measures as elements in addition to the condition information, the cluster, and the combination information in vector representation for each time, the second model outputting the combination information for the condition information and the cluster characteristic information.

### (Supplementary Note 6)

The information processing method according to any one of Supplementary Notes 1 to 5, comprising:
in a case where the types of measures each include a pair of a first measure belonging to a first hierarchy and a second measure belonging to a second hierarchy, separating a combination of a plurality of types of paired measures performed on the target person for each time, into first combination information representing a combination of the first measures belonging to the first hierarchy and second combination information representing a combination of the second measures belonging to the second hierarchy, classifying the first combination information as any one of a plurality of first clusters set in advance, and classifying the second combination information as any one of a plurality of second clusters set in advance;
generating the first model corresponding to the first hierarchy based on the condition information representing the condition of the target person and the first cluster as which the measures performed on the target person are classified for each time, the first model outputting the first cluster for the condition information;
generating the first model corresponding to the second hierarchy based on the condition information representing the condition of the target person and the second cluster as which the measures performed on the target person are classified for each time, the first model outputting the second cluster for the condition information; and
generating the second model based on the condition information, the first cluster, the second cluster, the first combination information, and the second combination information for each time, the second model outputting the first combination information and the second combination information for the condition information, information based on the first cluster, and information based on the second cluster.

### (Supplementary Note 7)

The information processing method according to Supplementary Note 6, comprising
generating the second model based on the condition information, the first cluster, the second cluster, and a plurality of types of combinations of the paired first measure and second measure for each time, the second model outputting a plurality of types of combinations of paired measures for the condition information, information based on the first cluster, and information based on the second cluster.

### (Supplementary Note 8)

The information processing method according to any one of Supplementary Notes 1 to 7, comprising:
by inputting new condition information into the first model, outputting a new cluster; and
by inputting information based on the new cluster output from the first model and the new condition information into the second model, outputting new combination information.

### (Supplementary Note 9)

The information processing method according to Supplementary Note 4 or 5, comprising:
by inputting new condition information into the first model, outputting a new cluster; and
by inputting the cluster characteristic information of the new cluster output from the first model and the new condition information into the second model, outputting new combination information.

### (Supplementary Note 10)

An information processing apparatus comprising:
a clustering unit that classifies combination information representing a combination of a plurality of types of measures performed on a target person for each time, as any one of a plurality of clusters set in advance;
a first model generating unit that generates a first model based on condition information representing a condition of the target person and the cluster as which the combination of the plurality of types of measures performed on the target person is classified for each time, the first model outputting the cluster for the condition information; and
a second model generating unit that generates a second model based on the condition information, the cluster, and the combination information for each time, the second model outputting the combination information for information based on the condition information and the cluster.

### (Supplementary Note 11)

The information processing apparatus according to Supplementary Note 10, wherein
the clustering unit classifies the combination information as any one of the clusters a number of which is set to a smaller number than a number of the types of measures.

### (Supplementary Note 12)

The information processing apparatus according to Supplementary Note 10 or 11, wherein
the clustering unit classifies the combination information in vector representation that contains the types of measures as elements, as any one of the clusters in accordance with a characteristic on the vector representation.

### (Supplementary Note 13)

The information processing apparatus according to any one of Supplementary Notes 10 to 12, wherein
the second model generating unit generates the second model based on cluster characteristic information representing a characteristic of the cluster in addition to the condition information, the cluster, and the combination information for each time, the second model outputting the combination information for the condition information and the cluster characteristic information.

### (Supplementary Note 14)

The information processing apparatus according to Supplementary Note 12, wherein
the second model generating unit generates the second model based on cluster characteristic information in vector representation that represents a characteristic of the cluster and that contains the types of measures as elements in addition to the condition information, the cluster, and the combination information in vector representation for each time, the second model outputting the combination information for the condition information and the cluster characteristic information.

### (Supplementary Note 15)

The information processing apparatus according to any one of Supplementary Notes 10 to 14, wherein:
in a case where the types of measures each include a pair of a first measure belonging to a first hierarchy and a second measure belonging to a second hierarchy, the clustering unit separates a combination of a plurality of types of paired measures performed on the target person for each time, into first combination information representing a combination of the first measures belonging to the first hierarchy and second combination information representing a combination of the second measures belonging to the second hierarchy, classifies the first combination information as any one of a plurality of first clusters set in advance, and classifies the second combination information as any one of a plurality of second clusters set in advance;
the first model generating unit generates the first model corresponding to the first hierarchy based on the condition information representing the condition of the target person and the first cluster as which the measures performed on the target person are classified for each time, the first model outputting the first cluster for the condition information;
the first model generating unit generates the first model corresponding to the second hierarchy based on the condition information representing the condition of the target person and the second cluster as which the measures performed on the target person are classified for each time, the first model outputting the second cluster for the condition information; and
the second model generating unit generates the second model based on the condition information, the first cluster, the second cluster, the first combination information, and the second combination information for each time, the second model outputting the first combination information and the second combination information for the condition information, information based on the first cluster, and information based on the second cluster.

### (Supplementary Note 16)

The information processing apparatus according to Supplementary Note 15, wherein
the second model generating unit generates the second model based on the condition information, the first cluster, the second cluster, and a plurality of types of combinations of the paired first measure and second measure for each time, the second model outputting a plurality of types of combinations of paired measures for the condition information, information based on the first cluster, and information based on the second cluster.

### (Supplementary Note 17)

The information processing apparatus according to any one of Supplementary Notes 10 to 16, comprising
an output unit that outputs a new cluster with input of new condition information into the first model, and outputs new combination information with input of information based on the new cluster output from the first model and the new condition information into the second model.

### (Supplementary Note 18)

The information processing apparatus according to Supplementary Note 13 or 14,
an output unit that outputs a new cluster with input of new condition information into the first model, and outputs new combination information with input of the cluster characteristic information of the new cluster output from the first model and the new condition information into the second model.

### (Supplementary Note 19)

A non-transitory computer-readable storage medium storing a program comprising instructions for causing an information processing apparatus to perform processes to:
classify combination information representing a combination of a plurality of types of measures performed on a target person for each time, as any one of a plurality of clusters set in advance;
   generate a first model based on condition information representing a condition of the target person and the cluster as which the combination of the plurality of types of measures performed on the target person is classified for each time, the first model outputting the cluster for the condition information; and
generate a second model based on the condition information, the cluster, and the combination information for each time, the second model outputting the combination information for information based on the condition information and the cluster.

### Reference Signs List

10 information processing apparatus
11 input unit
12 clustering unit
13 first learning unit
14 second learning unit
15 output unit
16 data storing unit
17 model storing unit
20 data management apparatus
100 information processing apparatus
101 CPU
102 ROM
103 RAM
104 programs
105 storage device
106 drive device
107 communication interface
108 input/output interface
109 bus
110 storage medium
111 communication network
121 clustering unit
122 first model generating unit
123 second model generating unit

What is claimed is:

1. An information processing method comprising:
classifying combination information representing a combination of a plurality of types of measures performed on a target person for each time, as any one of a plurality of clusters set in advance;
generating a first model based on condition information representing a condition of the target person and the cluster as which the combination of the plurality of types of measures performed on the target person is classified for each time, the first model outputting the cluster for the condition information; and
generating a second model based on the condition information, the cluster, and the combination information for each time, the second model outputting the combination information for information based on the condition information and the cluster.

2. The information processing method according to claim 1, comprising
classifying the combination information as any one of the clusters a number of which is set to a smaller number than a number of the types of measures.

3. The information processing method according to claim 1, comprising
classifying the combination information in vector representation that contains the types of measures as elements, as any one of the clusters in accordance with a characteristic on the vector representation.

4. The information processing method according to claim 1, comprising
generating the second model using machine learning based on cluster characteristic information representing a characteristic of the cluster in addition to the condition information, the cluster, and the combination information for each time, the second model outputting the combination information for the condition information and the cluster characteristic information.

5. The information processing method according to claim 3, comprising
generating the second model using machine learning based on cluster characteristic information in vector representation that represents a characteristic of the cluster and that contains the types of measures as elements in addition to the condition information, the cluster, and the combination information in vector representation for each time, the second model outputting the combination information for the condition information and the cluster characteristic information.

6. The information processing method according to claim 1, comprising:
in a case where the types of measures each include a pair of a first measure belonging to a first hierarchy and a second measure belonging to a second hierarchy, separating a combination of a plurality of types of paired measures performed on the target person for each time, into first combination information representing a combination of the first measures belonging to the first hierarchy and second combination information representing a combination of the second measures belonging to the second hierarchy, classifying the first combination information as any one of a plurality of first clusters set in advance, and classifying the second combination information as any one of a plurality of second clusters set in advance;
generating the first model corresponding to the first hierarchy based on the condition information representing the condition of the target person and the first cluster as which the measures performed on the target person are classified for each time, the first model outputting the first cluster for the condition information;
generating the first model corresponding to the second hierarchy based on the condition information representing the condition of the target person and the second cluster as which the measures performed on the target person are classified for each time, the first model outputting the second cluster for the condition information; and
generating the second model based on the condition information, the first cluster, the second cluster, the first combination information, and the second combination information for each time, the second model outputting the first combination information and the second combination information for the condition information, information based on the first cluster, and information based on the second cluster.

7. The information processing method according to claim 6, comprising
generating the second model based on the condition information, the first cluster, the second cluster, and a plurality of types of combinations of the paired first measure and second measure for each time, the second model outputting a plurality of types of combinations of paired measures for the condition information, information based on the first cluster, and information based on the second cluster.

8. The information processing method according to claim 1, comprising:
by inputting new condition information into the first model, outputting a new cluster; and
by inputting information based on the new cluster output from the first model and the new condition information into the second model, outputting new combination information.

9. The information processing method according to claim 4, comprising:
by inputting new condition information into the first model, outputting a new cluster; and
by inputting the cluster characteristic information of the new cluster output from the first model and the new condition information into the second model, outputting new combination information.

10. An information processing apparatus comprising:
at least one memory storing processing instructions; and
at least one processor configured to execute the processing instructions to:
classify combination information representing a combination of a plurality of types of measures performed on a target person for each time, as any one of a plurality of clusters set in advance;
generate a first model based on condition information representing a condition of the target person and the cluster as which the combination of the plurality of types of measures performed on the target person is classified for each time, the first model outputting the cluster for the condition information; and
generate a second model based on the condition information, the cluster, and the combination information for each time, the second model outputting the combination information for information based on the condition information and the cluster.

11. The information processing apparatus according to claim 10, wherein the at least one processor is configured to execute the processing instructions to
classify the combination information as any one of the clusters a number of which is set to a smaller number than a number of the types of measures.

12. The information processing apparatus according to claim 10, wherein the at least one processor is configured to execute the processing instructions to
classify the combination information in vector representation that contains the types of measures as elements, as any one of the clusters in accordance with a characteristic on the vector representation.

13. The information processing apparatus according to claim 10, wherein the at least one processor is configured to execute the processing instructions to
generate the second model based on cluster characteristic information representing a characteristic of the cluster in addition to the condition information, the cluster, and the combination information for each time, the second model outputting the combination information for the condition information and the cluster characteristic information.

14. The information processing apparatus according to claim 12, wherein the at least one processor is configured to execute the processing instructions to
generate the second model based on cluster characteristic information in vector representation that represents a characteristic of the cluster and that contains the types of measures as elements in addition to the condition information, the cluster, and the combination information in vector representation for each time, the second model outputting the combination information for the condition information and the cluster characteristic information.

15. The information processing apparatus according to claim 10, wherein the at least one processor is configured to execute the processing instructions to:
in a case where the types of measures each include a pair of a first measure belonging to a first hierarchy and a second measure belonging to a second hierarchy, separate a combination of a plurality of types of paired measures performed on the target person for each time, into first combination information representing a combination of the first measures belonging to the first hierarchy and second combination information representing a combination of the second measures belonging to the second hierarchy, classify the first combination information as any one of a plurality of first clusters set in advance, and classify the second combination information as any one of a plurality of second clusters set in advance;
generate the first model corresponding to the first hierarchy based on the condition information representing the condition of the target person and the first cluster as which the measures performed on the target person are classified for each time, the first model outputting the first cluster for the condition information;
generate the first model corresponding to the second hierarchy based on the condition information representing the condition of the target person and the second cluster as which the measures performed on the target person are classified for each time, the first model outputting the second cluster for the condition information; and
generate the second model based on the condition information, the first cluster, the second cluster, the first combination information, and the second combination information for each time, the second model outputting the first combination information and the second combination information for the condition information, information based on the first cluster, and information based on the second cluster.

16. The information processing apparatus according to claim 15, wherein the at least one processor is configured to execute the processing instructions to
generate the second model based on the condition information, the first cluster, the second cluster, and a plurality of types of combinations of the paired first measure and second measure for each time, the second model outputting a plurality of types of combinations of paired measures for the condition information, information based on the first cluster, and information based on the second cluster.

17. The information processing apparatus according to claim 10, wherein the at least one processor is configured to execute the processing instructions to:
output a new cluster with input of new condition information into the first model; and
output new combination information with input of information based on the new cluster output from the first model and the new condition information into the second model.

18. The information processing apparatus according to claim 13, comprising wherein the at least one processor is configured to execute the processing instructions to:
output a new cluster with input of new condition information into the first model; and
output new combination information with input of the cluster characteristic information of the new cluster output from the first model and the new condition information into the second model.

19. A non-transitory computer-readable storage medium storing a program comprising instructions for causing an information processing apparatus to perform processes to:
classify combination information representing a combination of a plurality of types of measures performed on a target person for each time, as any one of a plurality of clusters set in advance;
generate a first model based on condition information representing a condition of the target person and the cluster as which the combination of the plurality of types of measures performed on the target person is classified for each time, the first model outputting the cluster for the condition information; and
generate a second model based on the condition information, the cluster, and the combination information for each time, the second model outputting the combination information for information based on the condition information and the cluster.

20. The information processing method according to claim 1, wherein:
the target person is a patient; and
the measure is a treatment,
the information processing method comprising:
determining a treatment for the patient by inputting the condition information of the patient, the cluster, and the combination information into the generated second model; and
outputting the determined treatment to support decision making on the treatment for the patient by a user.
